(19) United States
(12) Patent Application Publication
(10) Pub. No.: US 2022/0257514 A1
(43) Pub. Date: Aug. 18, 2022

(54) MONOSACCHARIDE-TAGGED NANO-LIPOSOME DRUG DELIVERY SYSTEM, THE MANUFACTURE AND USE FOR DRUG TARGETING DELIVERY THEREOF

(71) Applicant: National Yang Ming Chiao Tung University, Taipei (TW)

(72) Inventors: Chun-Liang LO, Taipei (TW); Lu-Yi YU, Taipei (TW); Yao-An SHEN, Taipei (TW); Shang-Yu HUNG, Taipei (TW)

(21) Appl. No.: 17/613,783

(22) PCT Filed: May 23, 2019
(86) PCT No.: PCT/CN2019/088142

§ 371 Date: Nov. 23, 2021

(51) Int. Cl. A61K 9/127; A61K 47/69; A61K 47/54; A61K 47/24; A61K 31/704; A61K 38/14; A61K 31/407; A61K 31/513; A61K 31/675; A61K 31/4745; A61K 33/243; A61K 31/282; A61K 31/337; A61K 31/519; A61P 35/00

(52) U.S. Cl. CPC A61K 9/1275; A61P 35/00; A61K 47/549; A61K 47/24; A61K 31/704; A61K 38/14; A61K 31/407; A61K 31/513; A61K 31/675; A61K 31/4745; A61K 33/243; A61K 31/282; A61K 31/337; A61K 31/519; A61K 47/6911

(57) ABSTRACT

The present invention relates to a monosaccharide-tagged nano-liposome, which is characterized that the targeting monosaccharide is conjugated to cholesterol and the monosaccharide-conjugated cholesterol is incorporate into the phospholipid bilayer. The nano-liposome of present invention exhibits the ability to carry the loaded drug to target cells, such as cancer cells and cancer stem cells in a tumor tissue, and may be internalized by endocytosis to produce direct cytotoxicity or suppress stemness gene expression, so as to avoid toxicity to normal cells and effectively improve the therapeutic effect of cancer clinical medication and radiation therapy.

### CROSS-REFERENCE TO RELATED APPLICATION

This application claims priority of PCT Application No. PCT/CN2019/088142, filed on May. 23, 2019.

### BACKGROUND OF THE INVENTION

### Technical Field of the Invention

The present invention relates to a nano-liposome drug delivery system. Particularly, it relates to a nano-liposome with a monosaccharide ligand tagged on its surface, wherein the monosaccharide ligand is bonded to cholesterol incorporated into the phospholipid bilayer membrane of the liposome.

### Background

Liposome is a vesicle with phospholipid bilayer membranes that can be used to deliver drug molecule. Liposome is highly biocompatible since it has the similar structure of biological cells which have phospholipid bilayer plasma membranes. Although, the liposome has been widely used in drug delivery system. But the liposome without the ability of active targeting cannot effectively transport the active drug (like anti-cancer drug) into affected region (like tumor), thus need to increase the dosage for achieving the expected therapeutic.

It has been attempted in the art to attach a certain recognition molecule (also known as a targeting ligand) to liposome, through which the ligand molecule specifically interacts with the corresponding receptor on the surface of the target cell to deliver drugs exclusively into the targeted area, such as tumor tissue. There are several known targeting ligands including sugar, vitamin, lectin, peptide hormone, antigen, antibody, and other proteins. For example, U.S. Pat. No. 7,070,801 attempts to selectively delivering liposomes to target tissues or cells by linking sugars on the surfaces of the liposomes. However, the sugars are bonded to the liposomes through linker proteins, such as human albumin.

U.S. Pat. No. 8,802,153 B2 discloses a selective drug delivery system which is a nanoparticle made of polymers, such as polyethylene glycol (PEG) and polylactic acid (PLA). The delivery system comprises an anticancer agent, paclitaxel, and the nanoparticle is covalently associated with a prostate specific membrane antigen (PSMA) aptamer (Apt), and the covalent association is mediated by a linker moiety, which is PEG, so that the Apt is exhibited at the outermost layer of the nanoparticle. p U.S. Pat. No. 8,747,891 B2 discloses a ceramide anionic liposome comprising hydrophilic chemotherapeutics, wherein the liposome comprises at least one PEG-modified neutral lipid (at least half of which is PEG (750) C8), at least one anionic lipid, one ceramide and cationic or neutral lipid. In p U.S. Pat. No. 8,747,891 B2, the ceramide anionic liposome must have a net negative charge under physiological pH conditions. Although the PEG modification of liposome can help the liposome increasing the stability and circulation time in the blood, the research results of recent years has showed that PEG interferes the binding of ligands on the liposome surface and markers on target cells.

US patent No: US 2017/0112800 A1 discloses a hydrophobic taxane (alcohol)-lipid covalent conjugate, which provides additional stabilization for liposomes by generating supramolecular assembly in the phospholipid bilayer and increases the intratumoral concentration of the drug, thereby increasing the therapeutic efficacy. However, in the application, there is no specific description of the relevant techniques for the preparation of liposomes with targeting function including how to conjugate the targeting ligands to cholesterol.

Nowadays, the main problem of cancer treatment is that many anticancer drugs are not cancer-specific and the development of drug resistance/radiation resistance of cancer stem cells during the treatment, which will result in increasing the dose of chemotherapy drugs/radiation exposure and improving the risk and possibility of harmful side effects on the patients.

Therefore, the present invention provides a cholesterol conjugated with a monosaccharide or derivative thereof, and also uses of the monosaccharide conjugated cholesterol for mixing with at least one phospholipid to prepare a monosaccharide-tagged nano-liposome. The monosaccharide-tagged nano-liposome can be used as a delivery vehicle for anticancer drugs (like ceramide) to prevent or treat the resistance of cancer stem cells against chemotherapeutic drugs.

### SUMMARY OF INVENTION

Based on the above purpose, the present invention provides a method of preparing a glucosamine-tagged nano-liposome carrying ceramide, which has the ability to target cancer cells and cancer stem cells, increase the intracellular effect of anticancer drug, suppress the stemness gene expression of cancer stem cell and improve the efficacy of clinical anticancer drugs or radiotherapy against cancer.

Therefore, one aspect of the present invention relates to a monosaccharide-tagged nano-liposome drug delivery system comprising at least a cholesterol conjugated with a monosaccharide and a phospholipid. The monosaccharide-tagged nano-liposome drug delivery system can also comprise an unmodified cholesterol. In one embodiment of the present invention, the monosaccharide-conjugated cholesterol is located in the bilayer membrane structure of the liposome and the monosaccharide is exposed on the surface of the liposome, wherein the nano-liposome drug delivery system targets glucose transporter 1 (GLUT1) highly expressed on the surface of cancer cells and cancer stem cells and is internalized into the cell through endocytosis. Therefore, the delivery system of the present invention can deliver drugs to cancer cells or cancer stem cells. In one embodiment, the nano-liposome has a size of about 80-150 nm and a zeta-potential of −10 to −45 millivolts.

In some embodiments, the phospholipid is a neutral lipid, which refers to any uncharged lipid or neutrally charged zwitterion lipid at physiological pH value. The neutral lipid includes, but not limited to, distearyl phospholipid choline (DSPC), distearyl phospholipid ethanolamine (DOPE), distearyl phospholipid ethanolamine (DSPE), dipalmite phospholipid choline (DOPC), dipalmitophospholipid choline (DPPC), cephalin, cerebroside, diglycerin and sphingomyelin, etc. Furthermore, the phospholipid can also be an anionic lipid, which refers to any lipid that has a negative charge at physiological pH. Examples of anionic lipids include (but are not limited to) double hexadecyl phosphate (DHDP), phosphoinositide (PI), phospholipid serine (PS) such as dimyristyl phospholipid serine (DMPS), dipalmitoyl phospholipid serine (DPPS), glycerol phosphate (PG) such as dimyristyl glycerol (DMPG), dioleyl phosphatidyl glycerol, dioleyl phosphatidyl glycerol (DOPG), dilauryl phospholipid glycerol (DLPG), dipalmitic phospholipid glycerol (DPPG), distearyl phospholipid glycerol (DSPG), phosphatidic acid (PA) such as dimyristyl phosphate (DMPA), dipalmitic phosphate (DPPA) and diphospholipid glycerol (DPG).

In other embodiments, the monosaccharide is a monosaccharide molecule that can be conjugated to cholesterol, such as glucose, fructose, galactose, mannose, or derivatives thereof, wherein glucose or glucose derivatives (for example, glucosamine) are preferred.

In one embodiment of the present invention, the monosaccharide-tagged nano-liposome drug delivery system further comprises an anticancer drug, including a hydrophilic anticancer drug or a hydrophobic anticancer drug. In one embodiment of the present invention, the nano-liposome drug delivery system carries at least one chemotherapeutic drug in its cavity.

The monosaccharide-tagged nano-liposome drug delivery system can also be combined with a drug embedded in the phospholipid bilayer of the delivery system to form a targeted therapeutic nano-liposome, wherein the drug is embedded in the phospholipid bilayer of the liposome. For example, in one embodiment, the monosaccharide-tagged nano-liposome is loaded with ceramide to prepare a monosaccharide-tagged ceramide nano-liposome, wherein the ceramide is embedded in the bilayer membrane structure of the liposome. In this embodiment, the nano-liposome can further contain other drugs in its cavity to become a targeted therapeutic liposome with multiple drugs loaded in the phospholipid bilayer and cavity.

In some embodiments, the targeted therapeutic nano drug liposome or ceramide nano-liposome carry at least one anticancer drug in a cavity of the liposome. Preferably, the drug includes, but not limited to, doxorubicin, epirubicin, bleomycin, mitomycin C, 5-fluorouracil, cyclophosphamide, camptothecin, cisplatin, carboplatin, oxaliplatin, paclitaxel, docetaxel, gemcitabine, vinorelbine, irinotecan, etoposide, vinblastine, pemetrexed, hydroxyurea, methotrexate, capecitabine, floxuridine, cabazitaxel, mitoxantrone, estramustine, curcumin, camptothecin-like derivatives SN-38 and any combination thereof

In other embodiments, the targeted therapeutic nano-liposome or ceramide nano-liposome prevents or treats cancer stem cell resistance against the anticancer drug.

In another aspect of the present invention, it relates to a method of preparing a monosaccharide-tagged nano-liposome, comprising: synthesizing a monosaccharide-conjugated cholesterol, mixing a phospholipid, the monosaccharide-conjugated cholesterol, a unmodified cholesterol depending on needs and a drug, and using film hydration method, solvent dispersion method, organic solvent injection method, surfactant method, film extrusion method, French-Press method, etc., to manufacture a single phospholipid bilayer liposome with a certain size.

In some embodiments, the phospholipids, the monosaccharide-conjugated cholesterol and the drug are mixed in a ratio of 42-70 mmole % of dipalmitoylphosphatidylcholine (DPPC), 20-28 mmole % of the monosaccharide-conjugated cholesterol, and 10-30 mmole % of ceramide. In another embodiment, the monosaccharide-conjugated cholesterol is glucosamine-conjugated cholesterol.

In another aspect of the present invention, it relates to a pharmaceutical composition, which is characterized in comprising: a monosaccharide-tagged drug delivery system targeted therapeutic nano-liposome loaded with anticancer drug, and a pharmaceutically acceptable substrate, carrier or excipient. Preferably, the pharmaceutical composition is used for cancer treatment, including (but not limited to) cancer stem cell therapy, drug-resistance cancer cell therapy, radiation-resistant cancer cell therapy and combinations thereof

In some embodiments, the anticancer drug is ceramide and/or a chemotherapeutic drug. The pharmaceutical composition and the pharmaceutically acceptable substrate, carrier or excipient can be manufactured into various dosage forms for different administration routes by known methods in the pharmaceutical field, for example (but not limited to), solution, drop, pill, lozenge, powder, emulsion, transdermal dressing, ointment, cream and medicated scaffold.

The pharmaceutically acceptable substrate, carrier or excipient is any material know in the pharmaceutical field. In some embodiments, the pharmaceutically acceptable substrate comprising polysaccharides, proteins, synthetic polymers or mixtures thereof.

### BREIF DESCRIPTION OF THE DRAWINGS

FIG. 1 is a transmission electron microscope (TEM) image of a glucosamine-tagged nano-liposome prepared by an embodiment of the present invention in a physiological environment. The liposome is stained with uranyl acetate (2 w.t. %). The scale bar is 100 nm.

FIG. 2 shows the stability of the glucosamine-tagged nano-liposome in PBS buffer measured by DLS (upper half of the figure) and TEM (lower half of the figure). The liposome is stained with uranyl acetate (2 w.t. %) after 35 days of storage. The scale bar is 100 nm.

FIG. 3A-3B are the 3D type and ortho type of confocal microscope images showing the internalization of the glucosamine-tagged nano-liposome into non-small lung cancer cell sphere, H1299 (FIG. 3A), and colon cancer cell sphere, DLD-1 (FIG. 3B).

FIG. 4A-4B show that the glucosamine-tagged nano-liposome loaded with ceramide enhances the deliverability of ceramide. FIG. 4A exhibits that A549 non-small lung cancer stem cell spheres treated with the glucosamine-tagged ceramide nano-liposomes for 12 hours show a higher uptake of the liposomes into the cell spheres and efficient accumulated in the deep area (hypoxia area). The scale bar is 50 μm. FIG. 4B depicts the results of flow cytometry showing that Cy5.5 glucosamine-tagged nano-liposomes are more effectively delivered into A549 non-small lung cancer stem cell spheres.

FIG. 5A-5B show the accumulation of glucosamine-tagged ceramide nano-liposomes in various organs and tumors of animals and tumor tissues in in vivo experiments. FIG. 5A shows the image of non-invasive live imaging system exhibiting that the Cy5.5 glucosamine-tagged nano-liposomes are effectively accumulated in tumor tissues and reduced in other organs. FIG. 5B depicts the image of the frozen section, 3D type and ortho type of confocal fluorescence microscope showing that the Cy5.5 glucosamine-tagged nano-liposomes are internalized into the tumor tissue and accumulated in hypoxia area effectively. The scale bar is 100 μm.

FIG. 6 shows that the glucosamine-tagged ceramide nano-liposomes effectively inhibit the cancer stem cell sphere formation of A549 non-small lung cancer stem cell. The scale bar is 400 μm.

FIG. 7A-7B show that the glucosamine-tagged ceramide nano-liposomes can selectively kill cancer cells and cancer stem cells. FIG. 7A shows the results of flow cytometry indicating that the treatment of glucosamine-tagged ceramide nano-liposomes induces a higher apoptosis percentage of A549 non-small lung cancer stem cell spheres. FIG. 7B shows the results of flow cytometry indicating that the glucosamine-tagged ceramide nano-liposomes can induce higher apoptosis percentages of A549 parental cancer cells and A549 cancer stem cells than free ceramide, and the apoptosis rate is higher in A549 cancer stem cells than in A549 parental cancer cells, but no effect on normal L929 fibroblasts.

FIGS. 8A-8B show the sensitivity of A549 cancer stem cells to anticancer drugs (10 μM cisplatin; 5 μM paclitaxel) and to radiotherapy (5 Gy and 10 Gy), which is increasing in the presence of the glucosamine-tagged ceramide nano-liposomes. On the contrary, the number of surviving A549 cancer stem cells treated with the glucosamine-tagged ceramide nano-liposomes and the shRNA of retinoblastoma protein (RB). Free ceramide: using free ceramide to treat A549 cancer stem cells; G5C3: using the glucosamine-tagged ceramide nano-liposomes to treat A549 cancer stem cells; G5C3+shRB: using the glucosamine-tagged ceramide nano-liposomes and the shRNA of RB to co-treat A549 cancer stem cells; comparing free ceramide group and G5C3 group with control group; comparing G5C3+shRB group with G5C3 group. (* or #: p<0.05; ** or ##: p<0.01)

FIGS. 9A-9B show that A549 cancer stem cells treated with the glucosamine-tagged ceramide nano-liposomes have lower cell migration and invasion ability comparing to control group cells, but it can be restored if RB is inhibited. Free ceramide: using free ceramide to treat A549 cancer stem cells; G5C3: using the glucosamine-tagged ceramide nano-liposomes to treat A549 cancer stem cells; G5C3+shRB: using the glucosamine-tagged ceramide nano-liposomes and shRNA of RB to co-treat A549 cancer stem cells; comparing free ceramide group and G5C3 group with control group; comparing G5C3+shRB group with G5C3 group. (* or #: p<0.05; ** or ##: p<0.01)

FIG. 10A-10B indicate that the treatment of the glucosamine-tagged ceramide nano-liposomes combining with carboplatin/paclitaxel can inhibit in vivo tumor growth. FIG. 10A shows the relative tumor volume differences in mice, indicating that the therapeutic effect of the glucosamine-tagged ceramide nano-liposomes is equivalent to the clinical anticancer drug. Furthermore, the co-treatment of the nano-liposome of the present invention and the clinical anticancer drug can significantly enhance the therapeutic efficacy. FIG. 10B shows the weight changes of mice during the treatment, indicating that there is no significant side effect of the nano-liposomes.

FIG. 11 shows the H&E, Ki67 and caspase 3 stained sections of tumors treated with the combination of the glucosamine-tagged ceramide nano-liposomes and the carboplatin/paclitaxel, indicating that the combination of the glucosamine-tagged ceramide nano-liposomes and the carboplatin/paclitaxel can effectively cause tissue necrosis in tumor tissue and inhibit tumor proliferation. The scale bar is 200 μm.

FIG. 12A-12B are transmission electron microscope images of a glucosamine-tagged nano-liposome loading cisplatin prepared by an embodiment of the present invention showing that the nano-liposome has a spherical structure of phospholipid bilayer membrane. FIG. 12A is about the glucosamine-tagged ceramide nano-liposome loading cisplatin. FIG. 12B is about the glucosamine-tagged nano-liposome loading cisplatin. The scale bar is 100 nm.

FIG. 13 is a transmission electron microscope image of a glucosamine-tagged ceramide nano-liposome loading docetaxel prepared by an embodiment of the present invention showing that the nano-liposome has a spherical structure of phospholipid bilayer membrane. The liposome is stained with uranyl acetate (2 w.t. %). The scale bar is 100 nm.

### DETAILED DESCRIPTION OF THE INVENTION

Other features and advantages of the present invention will be further exemplified and described in the following embodiments. These exemplary embodiments are auxiliary explanations instead of the limitation to the scope of the present invention.

### EMBODIMENT 1. PREPARATION OF MONOSACCHARIDE-TAGGED NANO-LIPOSOME

### Synthesis of Glucosamine-cholesterol

The preparation process is as follows:

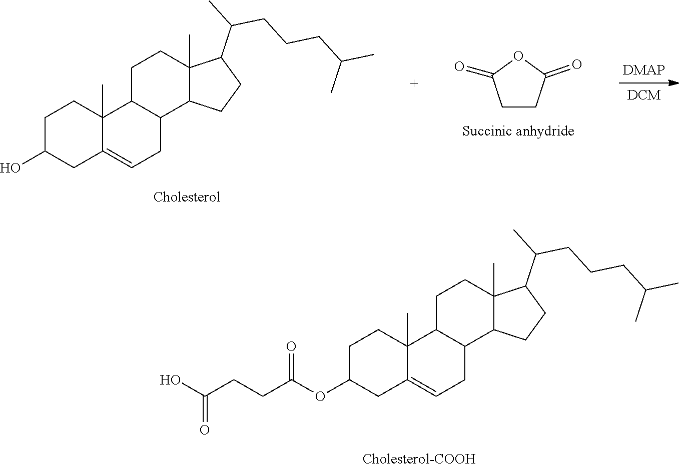 [SMILES: CC(C)CCCC(C)C1CCC2C3CC=C4CC(O)CCC4(C)C3CCC12C ; O=C1CCC(=O)O1 ; CC(C)CCCC(C)C1CCC2C3CC=C4CC(OC(=O)CCC(=O)O)CCC4(C)C3CCC12C]

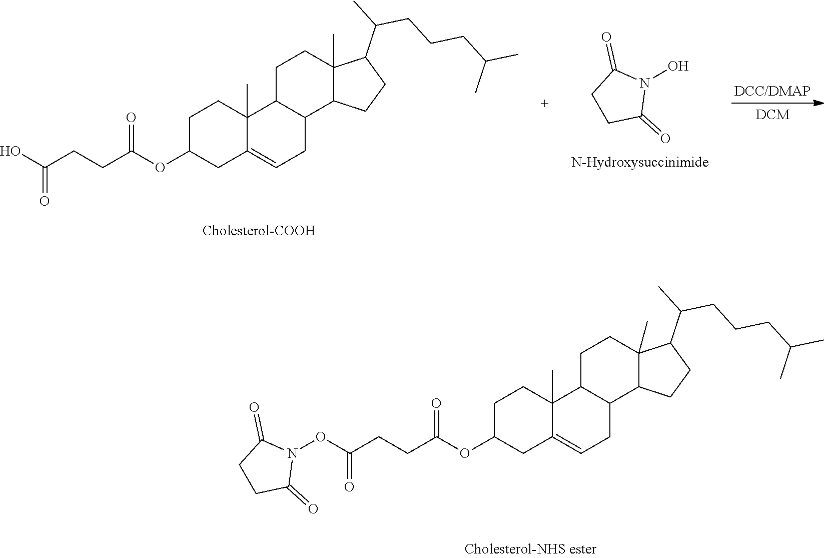 [SMILES: CC(C)CCCC(C)C1CCC2C3CC=C4CC(OC(=O)CCC(=O)O)CCC4(C)C3CCC12C ; O=C1CCC(=O)N1O ; CC(C)CCCC(C)C1CCC2C3CC=C4CC(OC(=O)CCC(=O)ON5C(=O)CCC5=O)CCC4(C)C3CCC12C]

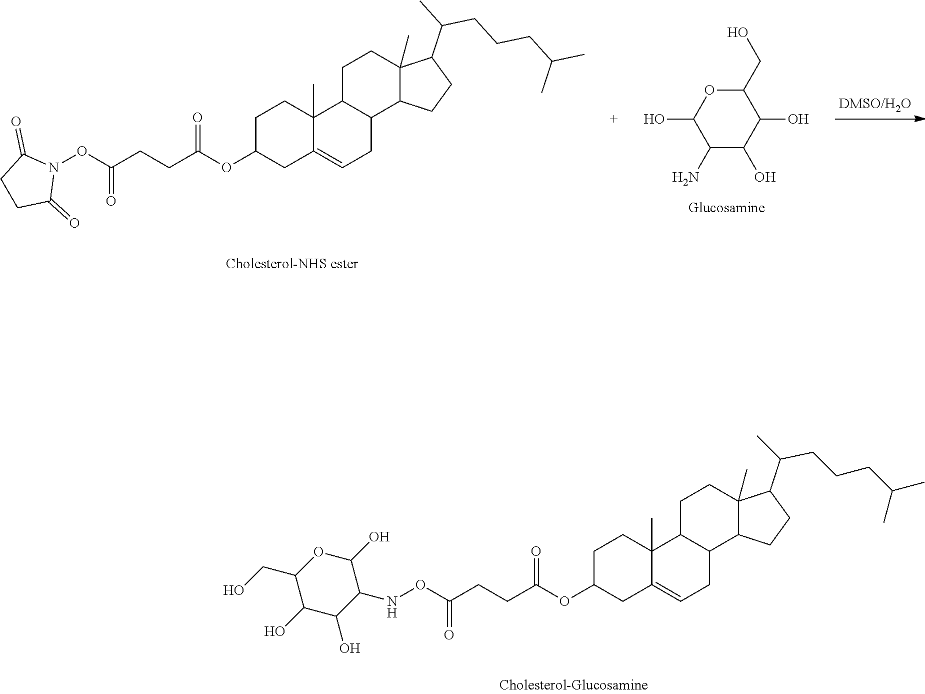 [SMILES: CC(C)CCCC(C)C1CCC2C3CC=C4CC(OC(=O)CCC(=O)ON5C(=O)CCC5=O)CCC4(C)C3CCC12C ; NC1C(O)OC(CO)C(O)C1O ; CC(C)CCCC(C)C1CCC2C3CC=C4CC(OC(=O)CCC(=O)ONC5C(O)OC(CO)C(O)C5O)CCC4(C)C3CCC12C]

Firstly, cholesterol-NHS esters are synthesized. Cholesterol (1 mmol), succinic anhydride (3 mmol), triethylamine (TEA, 1 mmol) and 4-dimethylaminopyridine (DMAP, 0.3 mmol) are dissolved in dry dichloromethane (DCM) and stirred at room temperature under nitrogen for 24 hours. After that, the product (carboxy-cholesterol) is extracted three times by saturated NaCl solution. And carboxy-cholesterol is dissolved in DCM, which will be removed later by rotary evaporator.

Carboxy-cholesterol (1 mmol), N-hydroxysuccinimide (NHS, 1.5 mmol) and DMAP (0.3 mmol) are dissolved in dry DCM, and the solution is poured into a two-neck round-bottom flask with a magnetic stir bar. Nitrogen gas is introduced into the flask, and N,N-dicyclohexylcarbodiimide (DCC, 3 mmol) pre-dissolved in dry DCM is slowly and dropwise added into the carboxy-cholesterol solution (placed in 0° C. ice bath),and then reacted under nitrogen for 24 hours with stirring. After that, the product is filtered to remove the by-product DCU. Then, the cholesterol-NHS solution is extracted three times by saturated NaCl solution and removed DCM by rotary evaporator. In the obtained product easter cholesterol-NHS ester, the 1H NMR (chloroform-d): δ0.6-2.4 (m, from cholesterol), δ2.6 (d, —COO—CH₂CH₂—COO— from succinic acid), δ2.6 (d, —COO—CH₂CH₂—COO— from succinic acid), δ2.843 (s, —CH₂—CH₂— from NHS), δ4.6-4.7 (s, —CH—O— from cholesterol), δ5.4 (s, —C═CH— from cholesterol).

Glucosamine (1.2 mmol) and the obtained cholesterol-NHS ester are dissolved in dimethyl sulfide (DMS6)/deionized water (volume ration is 1: 1) and placed in a glass bottle. After 24 hours of reaction, the product is extracted three times with saturated NaCl solution. And the glucosamine-cholesterol is dissolved in DCM, which will be removed later by rotary evaporator. In the final product, the 1H NMR (DMSO-d6): δ0.6-2.4 (m, from cholesterol), δ2.6 (t, —COO-CH₂CH₂—COO— from succinic acid), δ3.3-3.7 (s, —CH₂— and —CH from glucose), δ4.6-4.7 (m, —CH—O— from cholesterol), δ5.4 (m, —C═C— from cholesterol). The final product is also analyzed by SHIMADZY Fourier Transform Infrared Spectrometer (KBr): 1176 cm−1 (C—O—C elongation), 1258 cm−1 (O—C elongation), 1707 cm−1 (C═O elongation), 1793 cm−1 (C═O elongation), 2700-2900 cm−1 (C—H elongation), 2500-3300 cm−1 (O—H elongation).

### Preparation of the Glucosamine-tagged Nano-liposomes

The synthesized glucosamine-cholesterol (Glu-Chol), unmodified cholesterol, cholesterol with modified function group (—NH₂) (Chol-NH₂) and dipalmitophospholipid choline (DPPC) are dissolved in dichloromethane (DCM) with a mole ratio of 6.3:1.7:4.3:18.2, and then it is made into a liquid film by rotary evaporator at room temperature. After that, a 60° C. aqueous phosphate buffer solution (PBS) (pH 7.4) is added to rehydrate the film, resulting into a solution which will be subjected to ultrasonic vibration (22000 Hz) for 6 minutes. Later, the solution is passed through a 0.22-μm PVDF membrane (Millipore, Darmstadt, Germany) twice and a 0.1-μm PVDF membrane (Millipore, Darmstadt, Germany) twice to obtain liposomes tagged with different concentration of glucosamine. After reacting with Cy5.5-NHS ester aqueous solution for 2 days, it will be further dialyzed in MW6-8000 dialysis bag to remove unreacted Cy5.5 and obtain fluorescently labeled liposomes.

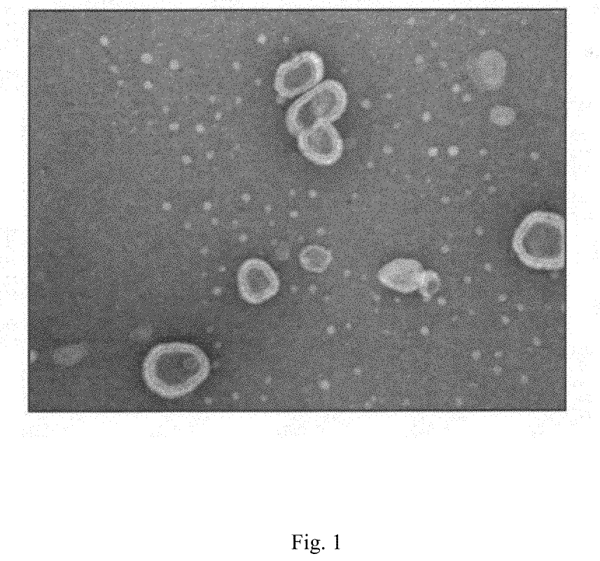

The glucosamine-tagged ceramide nano-liposome is stained with 2% uranyl acetate and observed by JEOL JEM-2000EX II transmission electron microscope (JEOL Inc., Peabody, MA). As showed in the transmission electron microscope image of FIG. 1, the nano-liposome of the present invention can maintain a good and complete morphology, a spherical structure with a phospholipid bilayer membrane, under physiological environment.

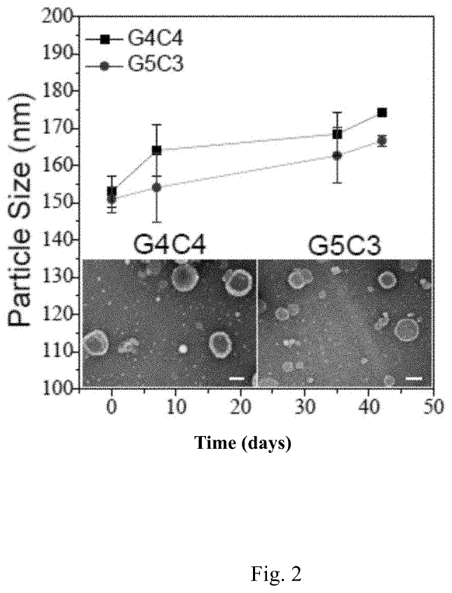

After leaving the nano-liposomes of the present invention in 4° C. PBS for 7, 35 and 42 days, the particle size and the changes of it are measured by DSL. And on the day 35, the nano-liposomes are stained with 2% uranyl acetate and observed by TEM microscopy to evaluate the stability of nano-liposomes. The results show that the particle size and shape of the ceramide nano-liposomes of the present invention remain stable after it has been stored at 4° C. for more than one month (FIG. 2).

In this embodiment, six groups of liposomes with different concentrations of glucosamine are prepared according to the liposome compositions listed in Table 1. Based on the analytical values in Table 1, the six groups of liposomes are similar in nature. The measurement of particle size, PDI, zeta-potential (zeta) is 120 nm, 0.2 and -3 to -15, respectively. Therefore, the ability to target the hypoxia area of tumor will be further examined.

TABLE 1
Particle size, zeta-potential and polydispersity index
(PDI) of the nano-liposomes with different compositions

| | | | | | | | | zeta- |
|---|---|---|---|---|---|---|---|---|
| | compositions (mmol) | | | | Particle | | | poten- |
| | | Glu- | Chol- | Choles- | size | | | tial |
| No. | DPPC | Chol | NH₂ | terol | (nm) | PDI | Kcps | (mV) |
| 1 | 18.2 | 0 | 1.7 | 15.1 | 126.6 | 0.219 | 346.2 | −3.79 |
| 2 | 18.2 | 1.3 | 1.7 | 12.9 | 129.3 | 0.229 | 301.5 | −11.9 |
| 3 | 18.2 | 2.5 | 1.7 | 10.8 | 125.8 | 0.219 | 339.6 | −9.22 |
| 4 | 18.2 | 3.8 | 1.7 | 8.6 | 128.6 | 0.214 | 382.2 | −10 |
| 5 | 18.2 | 5 | 1.7 | 6.5 | 132.6 | 0.222 | 369.3 | −12.7 |
| 6 | 18.2 | 6.3 | 1.7 | 4.3 | 123.6 | 0.22 | 282.3 | −7.9 |

The distribution of the glucosamine-tagged nano-liposomes in non-small lung cancer cell, H1299, and colon cancer cell sphere, DLD-1, is further discussed. The experimental method is briefly described as follows: the Cy5.5-fluorescence-labed nano-liposomes prepared as described above are co-cultured with non-small lung cancer tumor sphere or colon cancer tumor sphere for 5 hours, and then 150 μM pimonidazole (HP-1 hypoxia zone indicator) is added and incubated it for 1 hour. After that, the cells are fixed by formalin and immunostained with 1: 100 diluted FITC-mAb1. The cells will be observed under a confocal laser scanning microscopy (CLSM, Zeiss 880) to see the distribution of hypoxia zone indicator fluorescence. Also, the distribution of fluorescent dye labeled nano-liposomes in tumor tissue is examined by CLSM.

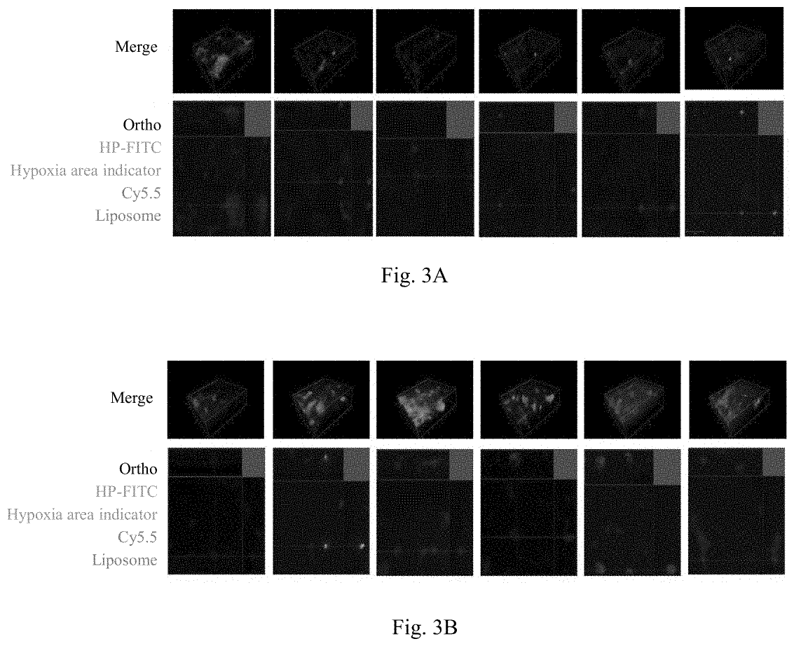

As shown in FIG. 3, the confocal laser scanning microscope images indicates that the amount of glucosamine-tagged nano-liposomes increases as the increasing concentration of glucosamine. In addition, the overlapping images of HP-FITC hypoxia zone indicator and fluorescent labeled Cy5.5-glucosamine-tagged nano-liposomes shows that the glucosamine-tagged liposome can target tumors and accumulate in the hypoxia zone of tumors, showing a significant difference when the concentration of glucosamine-cholesterol is higher than 2.5 mmole.

### EXAMPLE 2. THE PREPARATION OF THE GLUCOSAMINE-TAGGED CERAMIDE NANO-LIPOSOMES AND THE EVALUATION OF THE ABILITY TO TARGET CANCER CELLS AND CANCER STEM CELLS THEREOF

Preparation: the synthesized glucosamine-cholesterol, anticancer drug ceramide and dipalmitophospholipid choline (DPPC) are dissolved in dichloromethane (DCM) with a mole ratio of 10.9:4.1:3.5, and then it is made into a liquid film by rotary evaporator at room temperature. After that, a 60° C. aqueous phosphate buffer solution (pH 7.4) is added to rehydrate the film, resulting into a solution which will be subjected to ultrasonic vibration (22000 Hz) for 6 minutes. Later, the solution is passed through a 0.22-μm PVDF membrane (Millipore, Darmstadt, Germany) twice and a 0.1-μm PVDF membrane (Millipore, Darmstadt, Germany) twice to obtain No: G5C3 liposomes, glucosamine-tagged ceramide nano-liposomes, wherein the “G” represents glucosamine, and the “C” represents ceramide. With different content ratio of glucosamine and ceramide, different ceramide nanoliposomes are prepared, as shown in FIG. 2.

TABLE 2
Particle size, zeta-potential and polydispersity index (PDI)
of the ceramide nano-liposomes with different compositions

| | compositions (mole ratio) | | | | | | Zeta- |
|---|---|---|---|---|---|---|---|
| | | | glucosamine- | | particle size | | potential |
| No: | DPPC | cholesterol | cholesterol | ceramide | (nm)^{a} | PDI^{a} | (mV)^{a} |
| G0C0 | 10.9 | 6.5 | 0.0 | 0.0 | 129.1 ± 0.9 | 0.247 ± 0.147 | −3.48 ± 0.65 |
| G0C3 | 10.9 | 6.5 | 0.00 | 3.5 | 135.7 ± 2.2 | 0.399 ± 0.042 | −0.21 ± 0.27 |
| G4C4 | 10.9 | 0 | 3.2 | 4.7 | 153.0 ± 4.3 | 0.299 ± 0.005 | −11.56 ± 0.96 |
| G5C3 | 10.9 | 0 | 4.1 | 3.5 | 99.7 ± 3.72 | 0.236 ± 0.003 | −40.85 ± 3.77 |

^{a}particle size, zeta-potential and polydispersity index (PDI) are measured by dynamic light scattering (DLS).

According to the analysis results of dynamic light scattering (DLS), in the ceramide nano-liposomes with different compositions ratios, those with glucosamine tag have a particle size of 100-150 nm (Table. 2) and a PDI value of 0.2, approximately. It indicates that the resulting ceramide nano-liposomes are uniform in size.

The instrument Malvern Zetasizeer 1000HSA (Malvern Instruments, Malvern, UK) is used to measure the zeta-potential of the nano-liposomes of the present invention at 25° C. The results indicate that the ceramide nano-liposomes with high content of glucosamine has a zeta potential of between −10 to −45 millivolts (mV) and a ceramide encapsulation efficiency of 97 wt %. After incubating the nano-liposomes of the present invention in 4° C. phosphate buffer solution for 7, 35 and 42 days, the particle size and the changes of it are measured by DLS. On the day 35, the nano-liposomes are stained with 2% uranyl acetate and observed by TEM microscope to evaluate the stability of liposomes. The results show that the particle size and shape of the ceramide nano-liposomes of the present invention remain stable after been stored at 4° C. for more than one month.

The evaluation of the ability of the ceramide nano-liposomes to target cancer cells and cancer stem cells

Cancer stem cells (CSC) is a type of undifferentiated cells with self-renewal ability. In this embodiment, an in vitro tumour sphere model of suspension cultured lung cancer cells is used to evaluate the ability of the nano-liposomes to target cancer cells and cancer stem cells. Briefly, 1×10⁴ surviving cells treated with drugs are seeded in a culture dish coated with soft agar which prevents the cells from attaching and makes the cells form a suspended sphere. After 10 days, the number of spheres will be counted.

The G5C3 nano-liposomes prepared in this embodiment are reacted with Cy5.5-NHS ester for one day. The extra Cy5.5-NHS ester will be removed by phosphate buffer solution dialysis to obtain Cy5.5-G5C3 nano-liposomes. The Cy5.5-G5C3 nano-liposomes are co-cultured with A549 non-small lung cancer stem cells tumour spheres for 5 hours, and then 150 μM Hypoxyprobe-1 (HP-FITC anaerobic zone indicator) is added before further 1 hour incubation. After that, the cells are fixed by formalin and immunostained with 1: 100 diluted FITC-mAb1. The cells will be observed under a confocal laser scanning microscopy (CLSM, Zeiss 880) to present the fluorescence distribution.

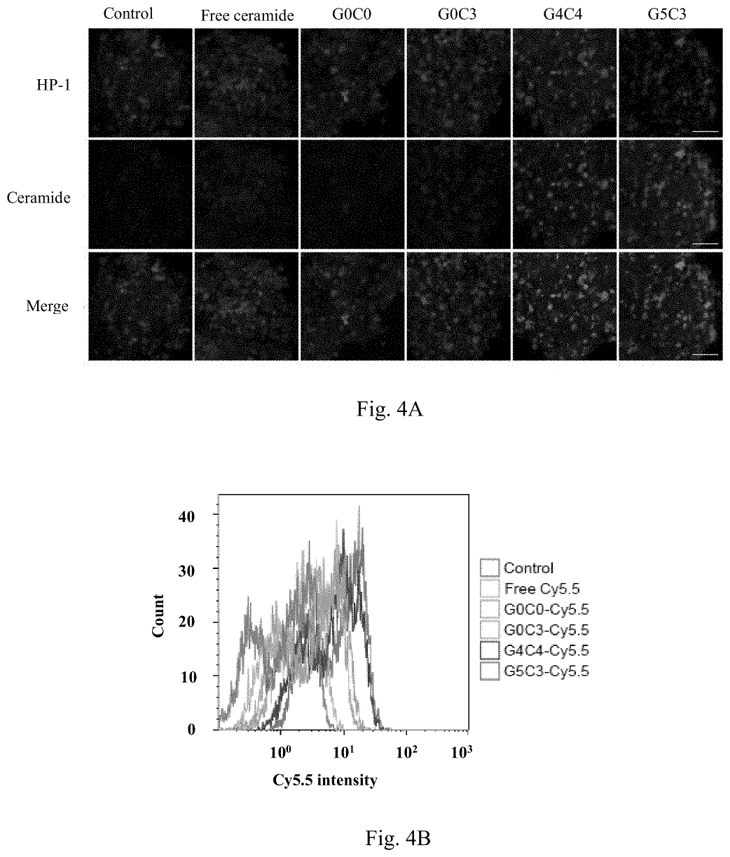

As shown in FIG. 4, the results of confocal laser scanning microscopy shows that the ceramide nano-liposome of the present invention (G4C4 and G5C3) can target the glucose transporter 1 (GLUT1) highly expressed on the surface of cancer cells or cancer stem cells by the glucosamine on the liposome membrane. And the liposomes are internalized into the cell via endocytosis to deliver the carried ceramide into cancer cells or cancer stem cells.

Nano-liposomes labeled with fluorescent dyes are prepared for in vivo tracking. The G5C3 nano-liposomes prepared in Example 1 is reacted with Cy5.5-NHS ester for one day, and the extra Cy5.5-NHS ester will be removed by phosphate buffer solution dialysis. H1299 cells (1×10⁷ cells/0.1 mL in Matrigel (Corning Matrigel Matrix, Corning)) are subcutaneously injected into the back surface of four-week-old female nude mice. After four weeks, the mice with H1299 tumors (tumor volume is around 500 mm³) are injected intravenously with 0.1 mL Cy5.5-G5C3 nano-liposomes (with dose of ceramide of 0.375 mg/kg⁻¹). After 23 hours, the mice with H1299 tumors are injected intraperitoneally with 0.1 mL hypoxyprobeTM-1 (concentration of 40 mg/mL). After another one hour, the mice are subjected to XENOGEN IVIS imaging system (IVIS50, PerkinElmer) to observe the in vivo distribution of Cy5.5-G5C3 nano-liposome.

The mice will be sacrificed, and the tumors and organs are collected. The tumor is fixed with formalin. The tissue section is embedded in Tissue-Tek O.C.T and immunostained with 1: 100 diluted FITC-mAb1. The fluorescence distribution is examined by confocal laser scanning microscope (CLSM, Zeiss 880) to present the distribution of nano-liposomes with fluorescent dye in tumor tissue.

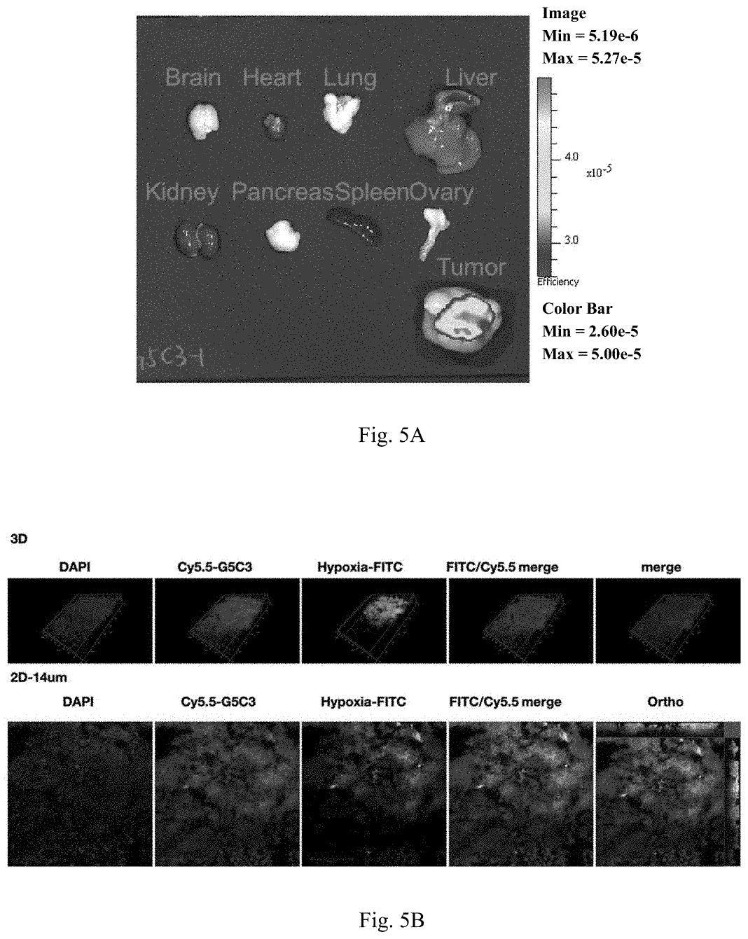

As shown in FIG. 5, G5C3 nano-liposomes mostly accumulate in tumor instead of other organs, such as brain and liver.

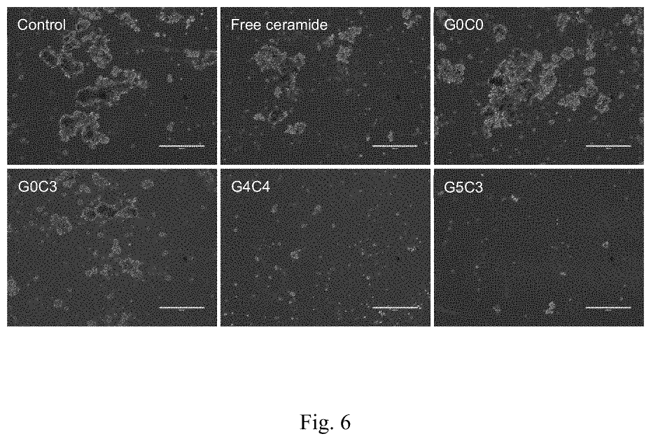

Furthermore, the formation of sphere is reduced in the groups treated with G4C4 and G5C3 nano-liposomes, which shows that the cancer stem cells in those treatment groups lose the ability of anti-apoptosis and long-term self-renewal (FIG. 6).

EXAMPLE 3. NANO-LIPOSOMES WITH SURFACE TAG OF CARBOHYDRATE CAN SELECTIVELY INDUCE APOPTOSIS OF LUNG CANCER CELL A549 CSC

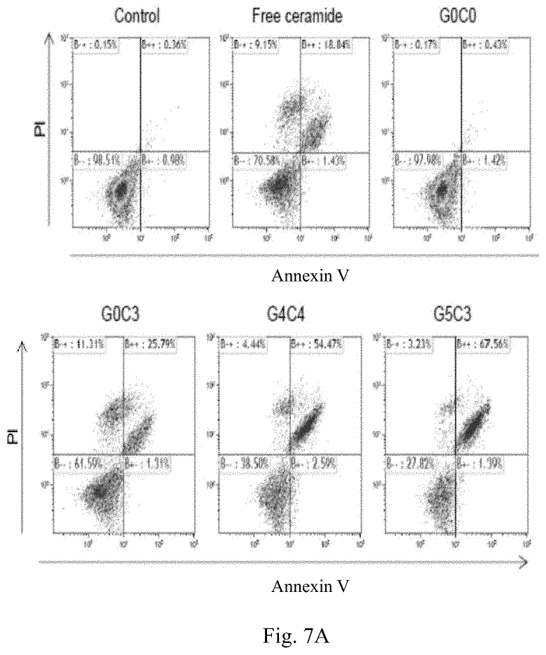

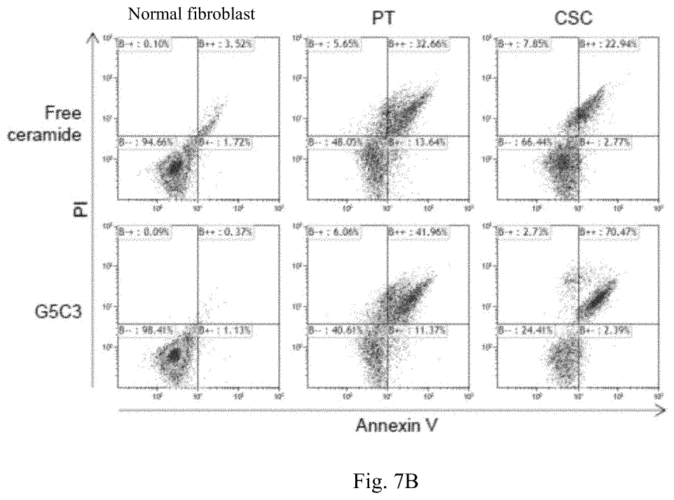

In this embodiment, Annexin V/PI staining is used to analyze the apoptosis-inducing effect of ceramide nano-liposomes of the present invention on cancer cells and cancer stem cells. The cells are stained with 5 μL annexin V-FITC and 5 μL propidium iodide (PI, 5 μg/ml) (BD Biosciences) in 1× binding buffer (10 mM HEPES, pH 7.4, 140 mM NaOH, 2.5 mm CaCl₂) at room temperature for 15 minutes. The cells are subjected to pass through Cytomics FC500 flow cytometer (Beckman Coulter) to evaluate the apoptosis by detecting the fluorescence of annexin V-FITC and PI. Those cells showing early apoptosis (annexin V+/PI−) and late apoptosis (annexin V+/PI+) are defined as dead cells. The results of Annexin-FITC/PI staining show that the nano-liposomes of the present invention enhance the delivery of ceramide. This corresponds to the results that the anti-apoptosis of the cell spheres is significantly inhibited by the treatment of G4C4 and G5C3 liposomes (FIG. 7A).

In order to prove that the glucosamine-tagged ceramide liposomes have selective cytotoxicity, the apoptotic effect of free ceramide and G5C3 nano-liposomes of the present invention on normal cell line (L929 fibroblasts) cultured under attached conditions is examined.

As shown in FIG. 7B, the results show that neither free ceramide nor G5C3 cause significant apoptosis in L929 fibroblasts. In parental lung cancer cells, the G5C3 nano-liposomes of the present invention have a higher intake rate and better cytotoxicity than free ceramide. It is known that A549 CSC is resistant to free ceramide; however, the CSC cells treated with G5C3 show higher cytotoxicity, which might be because the CSC has higher energy requirements of glycolysis than the parental cells. In general, the glucosamine-tagged ceramide liposomes of the present invention have selective cytotoxicity and can broadly block the treatment resistance of CSC without harmful effects on normal fibroblasts.

### EXAMPLE 4. EVALUATION OF THE INHIBITORY EFFECTS ON CANCER STEM CELL (CSC) BY GLUCOSAMINE-TAGGED NANO-LIPOSOMES COMBINED WITH CLINICAL ANTICANCER DRUGS/RADIOTHERAPY

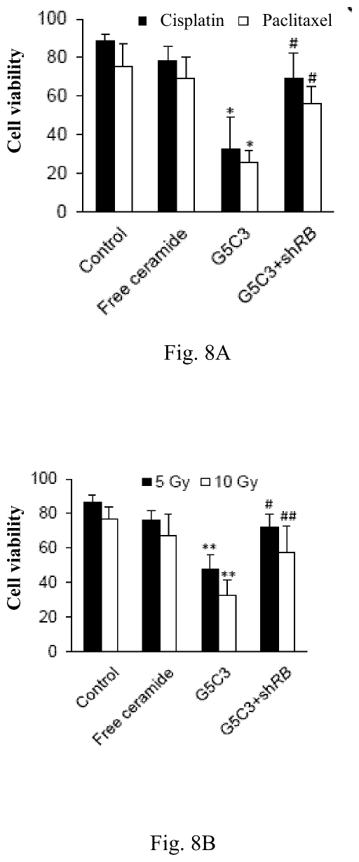

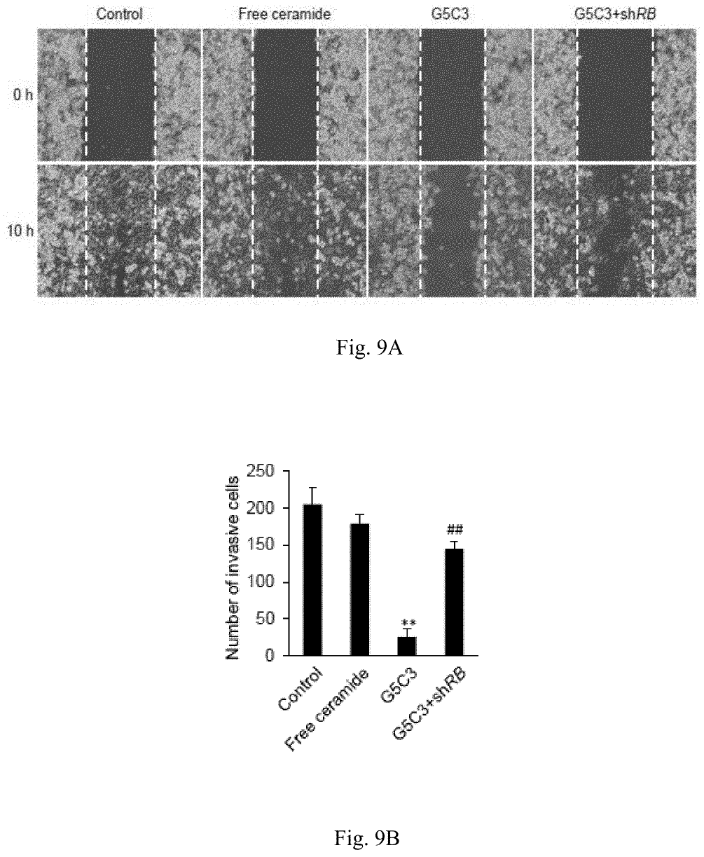

Cisplatin and paclitaxel, two clinical drugs commonly used in lung cancer treatment, are used to verify whether the drug resistance of lung cancer CSCs will be affected by the co-administration of G5C3 liposomes. Referring to the results of FIG. 8A, the CSCs treated with G5C3 liposomes are more sensitive to cisplatin and paclitaxel than the control group, but this effect will be inhibited if RB activity is blocked. In addition, the survival rate of CSCs treated with or without G5C3 liposomes under different radiation doses shows that CSCs treated with G5C3 liposomes are more sensitive to radiation than control group (FIG. 8B). However, CSCs will regain resistance to radiotherapy if RB activity is blocked, even in the presence of the G5C3 liposomes. Furthermore, the G5C3 liposomes also inhibit the migration and invasion ability of lung cancer CSC (FIG. 9A and 9B), but the reduced RB activity can increase the metastatic potential of CSCs, indicating that inhibiting the expression and activity of RB can counter act the effect of G5C3 on CSCs. That is, the differentiation state and reduced CSC characteristics caused by G5C3 is an RB-dependent. Based on the above results, the combination of G5C3 liposomes and clinical anticancer drugs/radiotherapy can synergistically inhibit the survival of CSCs, which helps to eliminate or reduce the resistance of CSCs to anticancer drugs/radiotherapy and improve the therapeutic effect.

### EXAMPLE 5. IN VIVO TUMOR SUPPRESSION EVALUATION OF THE GLUCOSAMINE-TAGGED NANO-LIPOSOMES COMBINED WITH ANTICANCER DRUGS/RADIOTHERAPY

In vivo tumour xenograft model is used to evaluate the in vivo tumor suppressive efficacy of the ceramide nano-liposomes combined with anticancer drugs. H1299 CSCs and H1299 cancer cells (1×10⁶ cells/0.1 mL) are injected with Matrigel (high concentration Matrix) into the back surface of four-week-old female nude mice for subcutaneous transplantation. One month later, the mice with H1299 tumor (tumor volume is around 100 mm³) are injected intravenously with carboplatin/paclitaxel (CP), the G5C3 ceramide nano-liposomes and the combination of carboplatin/paclitaxel and the G5C3 ceramide nano-liposomes (the dosage of each drug is 50 mg/kg carboplatin, 18 mg/kg paclitaxel and 0.375 mg/kg ceramide). The tumor volume (V) is measured by vernier calipers every two days for 30 days to evaluate the anti-tumor activity of drugs, and V=a×b²/2, wherein “a” and “b” are the long axis and short axis of the tumor, respectively. At the 30th day after injection, the mice are sacrificed and whole blood is collected for blood cell analysis, and the biochemical index is evaluated by using an automatic clinical chemistry analyzer (DRI-CHEM 4000i, FUJI) and a blood analyzer (XT-1800iv, Sysmex).

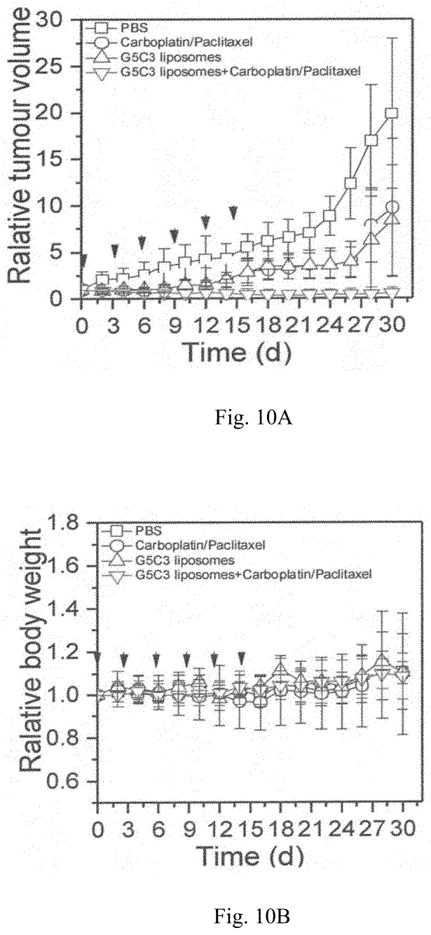

The results show that the combination of carboplatin/paclitaxel (CP) and G5C3 nano-liposomes has the most significant effect in inhibiting tumor growth (FIG. 10A). After 26 days, the anti-tumor ability of the combination of carboplatin/paclitaxel (CP) and G5C3 nano-liposomes is 9.6 times and 9.1 times than CP and G5C3 liposomes. And the results of in vivo administration indicate that G5C3 liposomes have strong anti-tumor ability and enhance the sensitivity of chemotherapy. Furthermore, the G5C3 nano-liposomes or the combination thereof with carboplatin/paclitaxel does not significantly change the body weight of mice (FIG. 10B), meaning that G5C3 can retain normal cells while having adverse impact on lung cancer cells without significant side effects on animal.

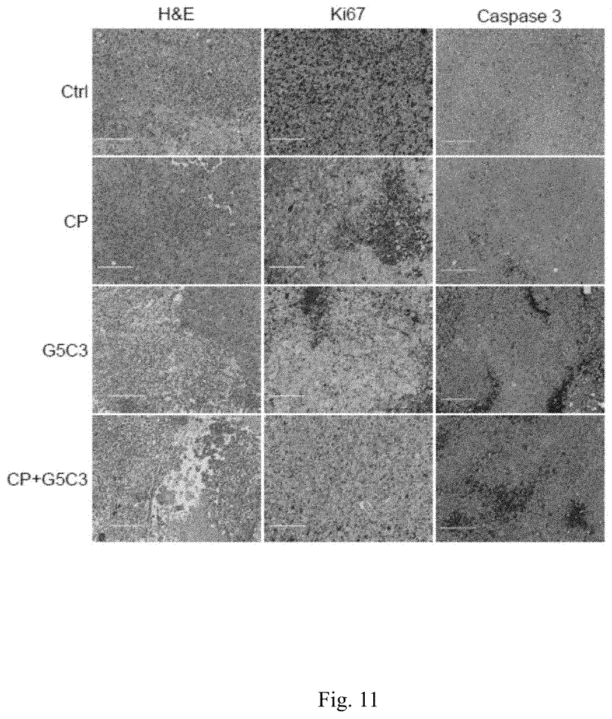

In this example, tumor tissues are histochemical stained with H&E, Ki-67 and caspase 3 for optical microscopy examination of tumor necrosis, proliferation and apoptosis evaluation. As shown in FIG. 11, the staining results of tumor tissue sections indicate that the tumor tissue treated with CP or G5C3 exhibits mild cell necrosis, and the tumor tissue treated with the combination of CP and G5C3 exhibits significant cell necrosis. And the histopathological results of Ki-67 staining show that the tumors in the control group have cell proliferation, and the treatment of CP combined with G5C3 nano-liposomes can significantly reduce the proliferation of cancer cells compared with the tumors only treated with CP or G5C3 nano-liposomes.

Furthermore, the histopathological results of caspase 3 staining show that the G5C3 nano-liposomes treatment and the combination treatment of CP and G5C3 nano-liposomes can significantly promote cell apoptosis compared with the CP treatment. Therese histopathological staining results of tumors are consistent with the results of the in vivo anti-tumor efficacy, suggesting that the combination of the nano-liposomes of the present invention and clinical anticancer drugs can effectively inhibit tumor proliferation, reduce the tumor volume, and even eliminate the tumor.

EXAMPLE 6. PREPARATION OF GLUCOSAMINE-TAGGED CERAMIDE NANO-LIPOSOMES WITH CISPLATIN

Dipalmitole phospholipid choline (DPPC), glucosamine-cholesterol prepared in Example 1, cholesterol and ceramide are added into a flask with the mole ratio listed in Table 3 and mixed with DCM. A rotary evaporator is used to remove DCM to form a thin film at the bottom of the flask, which will be left in a vacuum oven for one day. After that, 9 mL diethyl ether is added to dissolve the film at 40° C., and then 3 mL phosphate buffer solution containing cisplatin (oil: water=3: 1 v/v) is added at 60° C., following with vortex mixing. The diethyl ether is removed by rotary evaporator, and appropriate amount of phosphate buffer solution is added to the flask. The flask is left in oven at 60° C. for an hour. Finally, the mixture is filtered with 0.2 μm and 0.1 μm filter to obtain the glucosamine-tagged ceramide nano-liposomes loading cisplatin, named as GC-PL. The glucosamine-tagged nano-liposomes loading cisplatin are named as G-PL; the glucosamine-tagged ceramide nano-liposomes are named as GC-L; the glucosamine-tagged nano-liposomes are named as G-L; the ceramide nano-liposomes loading cisplatin are named as C-PL; and the nano-liposomes loading cisplatin are named as PL; wherein “G” represents glucosamine, “C” represent ceramide and “P” represent cisplatin. The composition of each nano-liposomes is shown in Table 3.

TABLE 3
Particle size, zeta potential and polydispersity index (PDI) of
nano-liposomes with or without ceramide (C) and/or cisplatin (P)

| | Compositions of liposomes (mole ratio) | | | | | | | zeta |
|---|---|---|---|---|---|---|---|---|
| | | | Glucosamine- | | | Particle size | | potential |
| Name | DPPC | Cholesterol | cholesterol | ceramide | cisplatin | (nm)^{a} | PDI^{a} | (mV)^{a} |
| GC-PL | 8.2 | 0 | 2.4 | 1.2 | 10 | 145.8 ± 8.5 | 0.123 ± 0.021 | −38.96 ± 7.98 |
| G-PL | 8.2 | 0 | 2.4 | 0 | 10 | 134.6 ± 4.6 | 0.125 ± 0.015 | −31.23 ± 1.83 |
| GC-L | 8.2 | 0 | 2.4 | 1.2 | 0 | 139.4 ± 0.9 | 0.109 ± 0.001 | −39.64 ± 6.77 |
| G-L | 8.2 | 0 | 2.4 | 0 | 0 | 134.3 ± 4.7 | 0.096 ± 0.019 | −35.54 ± 4.33 |
| C-PL | 8.2 | 3.9 | 0 | 1.2 | 10 | 244.3 ± 13.6 | 0.468 ± 0.098 | −19.52 ± 2.85 |
| PL | 8.2 | 3.9 | 0 | 0 | 10 | 207.3 ± 15.9 | 0.458 ± 0.037 | −22.90 ± 3.79 |

^{a}particle size, zeta-potential and polydispersity index (PDI) are measured by dynamic light scattering (DLS)

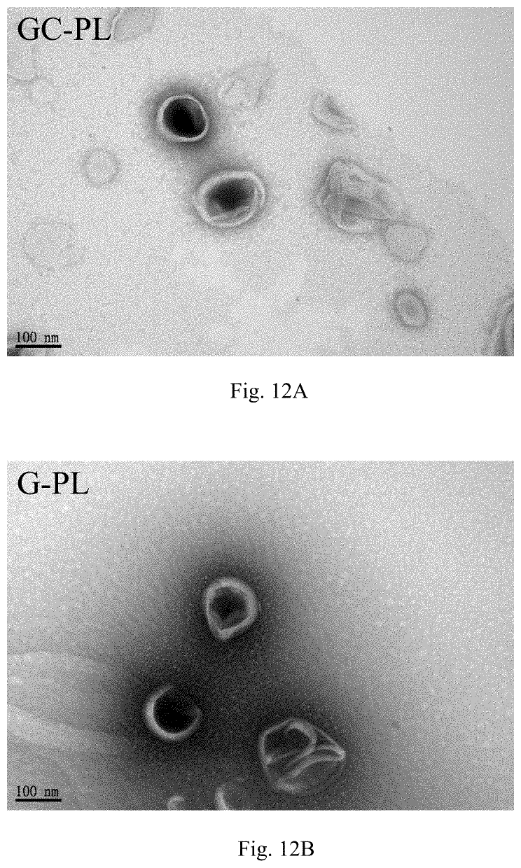

The nano-liposome is observed by JEOL JEM-2000EX II transmission electron microscope (JEOL Inc., Peabody, MA). As shown in FIG. 12 (the transmission electron microscope image), the glucosamine-tagged with or without ceramide nano-liposomes of the present invention can maintain a good and complete morphology, a spherical structure with a phospholipid bilayer membrane, under physiological environment.

The drug loading (DL) and encapsulation efficiency (EE) of the glucosamine-tagged ceramide nano-liposomes with cisplatin are further analyzed. To obtain a dry powder form of the liposomes, the liposome solution is concentrated and centrifuged to remove the unwrapped drug, and freeze-dried to remove water. 2 mg of the dry powder is added into a microcentrifuge tube for determining the platinum (Pt) content by inductively coupled plasma mass spectrometry (ICP-MS), so that the drug loading (DL) and encapsulation efficiency (EE) can be calculated.

The content and encapsulation efficiency (EE) of ceramide is evaluated by high performance liquid chromatography (HPLC). The liposome solution is concentrated and centrifuged to remove the unwrapped drug, and the residual volume is recorded. 1 ml of the liposome solution is added into a microcentrifuge tube and freeze-dried. Later, 1 ml of HPLC mobile phase is added for re-dissolving the dried powder, and the resulting solution is filtered. By using HPLC analysis, the amount of ceramide in 1 ml liposome solution is measured, helping to reckon the actual amount of ceramide to calculate the encapsulation efficiency. The wavelength for UV measurement is 230 nm, the flow rate is 1 ml/min, and the mobile phase is ACN/MeOH=3/7 (v/v). There is a ceramide signal at around 6 minutes. The weight of ceramide, which is deduced by calculating the area and introducing it into the calibration curve, can be used to reckon the drug loading (DL) and encapsulation efficiency (EE).

$$Drug loading = \frac{measured drug weight ( mg )}{weight of liposomes ( mg )} \times 100 \%$$

$$Encapsulaion efficiency = \frac{measured drug weight ( mg )}{drug weight of liposome in theory ( mg )} \times 100 \%$$

TABLE 4
Drug loading (DL) and encapsulation efficiency (EE) of nano-
liposomes with or without ceramide (C) and/or cisplatin (P)

| | |
|---|---|
| Ceramide | Cisplatin |

| Code | EE^{a}(w.t. %) | EE^{b}(w.t. %) | DL^{b}(w.t. %) |
|---|---|---|---|
| GC-PL | 99.2 ± 0.7 | 70.2 ± 1.5 | 26.7 |
| G-PL | — | 63.5 ± 0.9 | 25.2 |
| GC-L | 98.1 ± 1.2 | — | — |
| G-L | — | — | — |
| C-PL | 97.5 ± 1.4 | 67.1 ± 0.9 | 25.4 |
| PL | — | 55.1 ± 0.8 | 22.2 |

^{a}EE of ceramide is calculated by HPLC.
^{b}DL and EE of cisplatin is calculated by ICP-MS.

According to the results of HPLC and ICP-MS, the nano-liposomes can effectively encapsulate hydrophilic drugs and hydrophobic drugs. The nano-liposomes have 99% of the encapsulation efficiency for hydrophobic ceramide and 70% of the encapsulation efficiency for hydrophilic cisplatin.

### EXAMPLE 7. PREPARATION OF THE GLUCOSAMINE-TAGGED CERAMIDE NANO-LIPOSOMES WITH DOCETAXEL

Diₚalmitole phospholipid choline (DPPC), glucosamine-cholesterol prepared in Example 1, cholesterol and ceramide are added into a condensation flask with the mole ratio listed in Table 5, and dissolved and mixed well in DCM. A rotary evaporator is used to remove the DCM to form a thin film at the bottom of the flask, which will be left in a vacuum oven for one hour. Prepare solvent (A), 5 ml PBS+1.5 ml docetaxel (EtOH/PBS=1:1 (v/v)) and solvent (B), 5 ml PBS, in advance, and preheat them to 60° C. 15 ml of diethyl ether is added to the flask, and the flask is ultrasonically shock until the film is evenly dispersed in ether. The solvent (A) is added and vortex for a few seconds, and then the diethyl ether is removed by a rotary evaporator. The solvent (B) is added, then left in a 60° C. oven for an hour. Finally, the solution is filtered by 0.22-μm PVDF filter (Millipore, Darmstadt, German) and 0.1-μm PVDF filter (Millipore, Darmstadt, German) twice to obtain glucosamine-tagged ceramide nano-liposomes loading docetaxel, named as DL; wherein “D” represents docetaxel.

TABLE 5
Composition of the glucosamine-tagged
ceramide nano-liposomes with docetaxel

| | composition (mole ratio) | | | | |
|---|---|---|---|---|---|
| | | | glucosamine- | | |
| No | DPPC | cholesterol | cholesterol | ceramide | docetaxel |
| DL 0.375 | 10.9 | 0 | 4.1 | 3.5 | 0.93 |
| DL 0.75 | 10.9 | 0 | 4.1 | 3.5 | 1.86 |
| DL 1.5 | 10.9 | 0 | 4.1 | 3.5 | 3.71 |

The encapsulation efficiency (EE) and the drug weight of docetaxel are evaluated by HPLC. The liposome solution is concentrated and centrifuged to remove the unwrapped drug, and the residual volume is recorded. 1 ml of the liposome solution is added into a microcentrifuge tube and freeze-dried. Later, 1 ml of HPLC mobile phase is added for re-dissolving the dried powder, and the solution is filtered. By using HPLC analysis, the amount of docetaxel in 1 ml liposome solution is measured, helping to reckon the actual amount of docetaxel to calculate the encapsulation efficiency. The wavelength for UV measurement is 274 nm, the flow rate is 1 ml/min, and the mobile phase is ACN/H20=3/1 (v/v). There is a docetaxel signal at around 6 minutes. The weight of docetaxel, which is deduced by calculating the area and introducing it into the calibration curve, can be used to reckon the drug loading (DL) and encapsulation efficiency (EE). The calculation formula is as described in Example 6.

TABLE 6
Particle size, zeta potential, polydispersity index (PDI), drug loading (DL), and encapsulation
efficiency of the glucosamine-tagged ceramide nano-liposomes with docetaxel

| | Particle size^{a} | | | Zeta^{a} | DL^{b} | EE^{b} |
|---|---|---|---|---|---|---|
| No | (nm) | PDI^{a} | Kcps^{a} | (mV) | (w.t. %) | (w.t. %) |
| DL 0.375 | 140.6 ± 4.6 | 0.150 ± 0.010 | 304.8 ± 134.2 | −23.6 ± 3.5 | 3.6 ± 1.0 | 61.2 ± 16.3 |
| DL 0.75 | 146.5 ± 14.1 | 0.140 ± 0.014 | 225.3 ± 16.9 | −30.8 ± 7.5 | 5.04 | 27.8 ± 15.7 |
| DL 1.5 | 145.1 ± 12.4 | 0.135 ± 0.020 | 223.5 ± 54.1 | −29.9 ± 5.3 | 7.21 | 26.9 ± 8.5 |

^{a}particle size, zeta-potential and polydispersity index (PDI) are measured by dynamic light scattering (DLS)
^{b}DL and EE of docetaxel is calculated by HPLC

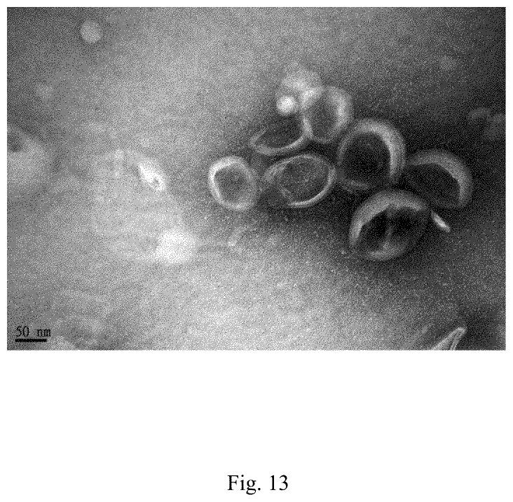

The nano-liposome is stained with 2% uranyl acetate and observed by JEOL JEM-2000EX II transmission electron microscope (JEOL Inc., Peabody, Mass.). As shown in the transmission electron microscope image (FIG. 13), the glucosamine-tagged docetaxel nano-liposomes with or without ceramide of the present invention can maintain a good and complete morphology, a spherical structure with a phospholipid bilayer membrane, under physiological environment.

In summary, the present invention firstly synthesizes a monosaccharide-tagged cholesterol, and uses the monosaccharide-tagged cholesterol to prepare monosaccharide-tagged nano-liposome drug delivery particles by mixing it with phospholipids, active drugs and optionally untagged cholesterol. As proved by the results of cell and animal experiments, the monosaccharide-tagged nano-liposomes of the present invention can specifically target and deliver the carried drugs to targeted cancer cells and cancer stem cells. And the nano-liposomes can be internalized into the target cells through endocytosis to directly cause cytotoxicity or suppress stemness gene expression. Therefore, the monosaccharide-tagged nano-liposomes can be effectively used in the preparation of targeted therapeutic nano-drugs. And it has been proved by in vivo administration experiments that the monosaccharide-tagged nano-liposomes of the present invention can effectively inhibit tumor growth and cancer metastasis without causing harmful side effects to the animal subject. And when the monosaccharide-tagged nano-liposomes is combined with clinical anticancer drugs/radiotherapy, it presents synergistic tumor suppression effect and prevents cancer stem cells from developing the resistance of anticancer drugs.

1. A monosaccharide-tagged nano-liposome drug delivery system, comprising at least a cholesterol conjugated with a monosaccharide and a phospholipid.

2. The monosaccharide-tagged nano-liposome drug delivery system according to claim 1, wherein the phospholipid is selected from a group consisting of distearyl phospholipid choline (DSPC), dioleyl phospholipid ethanolamine (DOPE), Distearyl phospholipid ethanolamine (DSPE), dipalmitophospholipid choline (DOPC), dipalmitophospholipid choline (DPPC), cephalin, cerebroside, diglycerin and sphingomyelin, double ten six-carbon chain phosphate (DHDP), phosphoinositide (PI), phospholipid serine (PS), dimyristyl phospholipid serine (DMPS), dipalmitoyl phospholipid serine (DPPS), Glycerol phosphate (PG), dimyristyl glycerol (DMPG), dioleyl phospholipid glycerol (DOPG), dilauryl phospholipid glycerol (DLPG), dipalmitophospholipid glycerol (DPPG), two Stearyl phospholipid glycerol (DSPG), phosphatidic acid (PA), dimyristyl phosphate (DMPA), dipalmitic phosphate (DPPA), diphospholipid glycerol (DPG) and mixtures thereof.

3. The monosaccharide-tagged nano-liposome drug delivery system according to claim 1, wherein the nano-liposome has a size of between 120-140 nm and a zeta potential of between −3 to −15 millivolt.

4. The monosaccharide-tagged nano-liposome drug delivery system according to claim 1, wherein the monosaccharide is selected from glucose, fructose, galactose, mannose or monosaccharide derivatives.

5. The monosaccharide-tagged nano-liposome drug delivery system according to claim 1, or wherein the monosaccharide is glucose or glucosamine.

6. The monosaccharide-tagged nano-liposome drug delivery system according to claim 1 further comprises an unmodified cholesterol.

7. The monosaccharide-tagged nano-liposome drug delivery system according to claim 1 further comprises an anticancer drug in a cavity of the liposome.

8. The monosaccharide-tagged nano-liposome drug delivery system according to claim 7, wherein the anticancer drug is selected from a group consisting of doxorubicin, epirubicin, bleomycin, mitomycin C, 5-fluorouracil, cyclophosphamide, camptothecin, cisplatin, carboplatin, oxaliplatin, paclitaxel, docetaxel, gemcitabine, vinorelbine, irinotecan, etoposide, vinblastine, pemetrexed, hydroxyurea, methotrexate, capecitabine, floxuridine, cabazitaxel, mitoxantrone, estramustine, curcumin, camptothecin-like derivatives SN-38 and any combination thereof

9. A method of preparing a monosaccharide-tagged nano-liposome drug delivery system according to claim 1, comprising:
synthesizing a monosaccharide-conjugated cholesterol;
mixing a phospholipid, the synthesized monosaccharide-conjugated cholesterol, and an appropriate unmodified cholesterol into a mixture; and
using a film hydration method, solvent dispersion method, organic solvent injection method, surfactant method, film extrusion method, or French-Press method to make the mixture into a single phospholipid bilayer liposome with a certain size.

10. The method according to the claim 9, wherein the phospholipid and the monosaccharide-conjugated cholesterol are mixed in a ratio of 50-60 mmole % of the phospholipid, 20-48 mmole % of the cholesterol and 2-20 mmole % of the monosaccharide-conjugated cholesterol.

11. A targeted therapeutic nano-liposome, comprising: a monosaccharide-tagged nano-liposome drug delivery system according to claim 1 and a drug embedded in the phospholipid bilayer of the liposome drug delivery system.

12. The targeted therapeutic nano-liposome according to claim 11, wherein the nano-liposome has a size of between 80-160 nm and a zeta potential of between −10 to −45 millivolt.

13. The targeted therapeutic nano-liposome according to claim 11, wherein the drug is ceramide.

14. A method for preparing a targeted therapeutic nano-liposome, comprising:
synthesizing a monosaccharide-conjugated cholesterol;
mixing a phospholipid, the synthesized monosaccharide-conjugated cholesterol, and a drug into a mixture; and
using a film hydration method, solvent dispersion method, organic solvent injection method, surfactant method, film extrusion method, or French-Press method to make the mixture into a single phospholipid bilayer liposome with a certain size.

15. The method according to claim 14, wherein the phospholipid, the monosaccharide-conjugated cholesterol and the drug are mixed in a ratio of 52-77 mmole % of the phospholipid, 17-23 mmole % of the monosaccharide-conjugated cholesterol and 6-25 mmole % of the drug.

16. A pharmaceutical composition, comprising the monosaccharide-tagged nano-liposome drug delivery system according to claim 1 or the targeted therapeutic nano-liposome and a pharmaceutically acceptable substrate, carrier or excipient.

17. The pharmaceutical composition according to claim 16, wherein the substrate is selected from polysaccharides, proteins, synthetic polymers or a mixture thereof.

18. The pharmaceutical composition according to claim 16, wherein the anticancer drug is selected from a group consisting of doxorubicin, epirubicin, bleomycin, mitomycin C, 5-fluorouracil, cyclophosphamide, camptothecin, cisplatin, carboplatin, oxaliplatin, paclitaxel, docetaxel, gemcitabine, vinorelbine, irinotecan, etoposide, vinblastine, pemetrexed, hydroxyurea, methotrexate, capecitabine, floxuridine, cabazitaxel, mitoxantrone, estramustine, curcumin, camptothecin-like derivatives SN-38 and any combination thereof.

19. The pharmaceutical composition according to claim 16, which is used for a cancer treatment.

20. The pharmaceutical composition according to claim 19, wherein the cancer treatment is selected from cancer stem cell therapy, drug-resistant cancer cell therapy, radiation-resistant cancer cell therapy and any combination thereof.
